(19) United States
(12) Patent Application Publication
(10) Pub. No.: US 2009/0270321 A1
(43) Pub. Date: Oct. 29, 2009

(54) Over-expression of MPS-1 gene or its gene product(s) results in reduction in size of a variety of malignancies

(76) Inventors: Brendan Curran Stack, JR., Roland, AR (US); Yuemeng Dai, Little Rock, AR (US)

Correspondence Address: Brendan C. Stack, Jr., MD, 21001 Lake Vista Dr., Roland, AR, 72135, US

(21) Appl. No.: 12/384,962

(22) Filed: Apr. 10, 2009

Related U.S. Application Data
(60) Provisional application No. 61/123,691, filed on Apr. 10, 2008.

(51) Int. Cl. A61K 38/16

(52) U.S. Cl. 514/12

(57) ABSTRACT

Metallopanstimulin-1 (MPS-1) is a multifunctional ribosomal protein. MPS-1 is a 10 kD zinc finger protein (ZFP) that is present in all tissues and expressed in increased quantities in a wide variety of proliferating tissues and malignancies. The ribosome is a ribonucleoprotein complex of ribosomal RNAs (rRNA) and proteins (r-protein). Conventionally, r-proteins are thought to be responsible for new protein synthesis, but emerging evidence has shown activity exists beyond this. Some extra-ribosomal functions of r-proteins have been observed in close relation to cancer pathology. MPS-1 is over-expressed in the serum and tissue of dozens of malignancies. However, it is unknown whether the enhanced expression of MPS-1 is the cause or result of tumor development and progression. Our preliminary observations show increased MPS-1 decreases paxillin. Recently, paxillin was found to have an important role in regulating both normal and tumor cell proliferation.

Elevation of MPS-1 in HNSCC samples and its structure/function as a ribosomal ZFP have led us to conclude that MPS-1 might be a subject of interest for malignant tumor therapy. Our data reveal that enhanced expression of MPS-1 protein can strongly suppress tumor cell proliferation. MPS-1 and significantly inhibits tumor growth both in vitro and in vivo in 3 distinct malignant cell lines. We have concluded that over-expression of MPS-1 might be used therapeutically in the treatment of some malignancies.

### STATEMENT OF GOVERNMENTAL INTEREST

None

### BACKGROUND OF THE INVENTION

Metallopanstimulin-1 (MPS-1) is the multifunctional ribosomal protein RPS27, a component of the 40S ribosomal subunit, and it contains a zinc finger domain of the C₄ type. MPS-1 was discovered by Fernandez-Pol from a cDNA library of a human mammary carcinoma cell line (MDA-468), stimulated by the growth factors TGF-β1 and EGF in the presence of cyclohexamide (DNA vector with isolated cDNA gene encoding metallopanstimulin, U.S. Pat. No. 5,243,041, Fernandez-Pol, Sep. 7, 1993). MPS-1 has been found highly expressed in a number of cancers, including HNSCC, (FIG. 1). MPS-1 is a 9.5 kD, 81 amino acid residue polypeptide present at low levels in all tissues and expressed in large quantities in cancers. MPS-1 distributes not only in the cytosol, but also in the nuclei. When MPS is over-expressed, it is either secreted or passively released down a concentration gradient into the extra-cellular space. However, little is known about the role of MPS-1 in regulating cancer cell behavior. MPS-1 is a zinc finger protein (ZFP). ZFPs have been described as “the next targets for anti-viral therapy” (NIH Federal Register 1995; 60; 154:40844-6, August 10).

Reported functions of zinc finger proteins include:
1. Protein synthesis.
2. Viral infections.
3. DNA binding.
4. Steroid binding.
5. RNA binding.
6. Internal reservoir or buffer of zinc and other divalent cations (Cu, Fe, Mg, Mn, etc.).
7. Many other functions have been ascribed to ZFPs that suggest a large and varied role in cellular biochemistry.

The ribosome is a ribonucleoprotein complex of ribosomal RNA (rRNA) and ribosomal protein (r-protein). The eukaryotic ribosome is composed of four rRNAs and about 80 r-proteins. Conventionally, the r-proteins are thought to be responsible for new protein synthesis, but emerging evidence has shown that r-proteins possess many other essential “extra-ribosomal functions”. In Drosophila, r-protein S3 (rpS3) was found to have DNase activity that specifically cleaves DNA containing an apurinic/apyrimidinic (AP) site via a β-elimination reaction, suggesting that rpS3 has a role in DNA repair. Drosophila rpS3 also contains DNA deoxyribophospho diesterase (dRpase) activity and plays an important role in the repair of oxidative and ionizing radiation-induced DNA damage. Moreover, rpS3 can induce mouse plasmacytoma MPC-11 B-cell apoptosis by activating caspase-8/caspase-3. Other research groups have shown that several other r-proteins, such as S29, S27L, S13, and L3, have functions of either promoting or suppressing apoptosis. The r-proteins also have development and proliferation regulation functions. For example, over-expression of rpS19 improves erythroid development and increases the number of erythroid colonies in rpS19-deficient Diamond Blackfan anemia.

It has been long recognized that many r-proteins are over-expressed in various cancer cell lines and primary tumors, but it is not known whether the over-expression of certain r-proteins is a cause of tumor development or a response to rapid cancer cell proliferation. Previous studies have demonstrated that MPS-1 protein is highly expressed in various types of tumors, and higher levels of MPS-1 are related to advanced cancer stage. Therefore, MPS-1 was thought to be involved in tumorigenesis. However, our current data support the notion that high level MPS-1 does not stimulate HNSCC tumor growth, but inhibits tumor growth.

Some extra-ribosomal functions of r-proteins have been observed in close relation to oncogenesis. Naora et al reported that enhancement of RPS3a expression induced transformation of NIH 3T3 cells and formation of tumors in nude mice. In addition, more r-proteins are elevated in various types of cancers, such as over-expression of RPS27a in colorectal carcinoma; enhanced expression of r-proteins S8, L12, L23a, L27 and L30 in hepatocellular carcinoma; and increased expression of RPL19 in prostate cancer. In our previous studies, we found that MPS-1, encoded by a TGF-β inducible gene, is over-expressed in HNSCC and that greater expression of MPS-1 in circulation is related to increased tumor burden

High levels of MPS-1 have also been observed in other various types of human cancers including prostate, colon, liver, breast, and gastric. However, it is not clear whether the MPS-1 at high levels alters tumor cell proliferation and tumor growth.

Many metalloproteins (e.g., MPS-1) are involved in DNA repair and promotion of ongoing cell growth and proliferation and are elevated in HNSCC. Chelation of the catalytic ion may be an alternative means of inducing cellular arrest, if not apoptosis. This may be accomplished by administration of supra-physiologic amounts of some naturally occurring carboxylic acids. Because MPS-1 is over-expressed in HNSCC (and other tumors) and may be an effecter molecule in oncogenesis, potential therapies directed towards MPS-1 and similar ZFPs may provide a tumorostatic or tumorocidal effect that may be a stand-alone novel therapy or combined with traditional therapies.

Paxillin is a key component of focal adhesion, and functions as a scaffold protein that facilitates assembly of multi-protein complexes to integrate and transmit signals. Recently, paxillin was found to have an important role in regulating both normal and tumor cell proliferation. Over-expression of paxillin can enhance growth of H522 lung-cancer cells in vivo compared with control vector-transfected H522 cells. Immunohistochemistry staining with Ki-67 antibody revealed an increased H522 cell proliferation rate after paxillin transfection. Knockdown of paxillin by siRNA in mouse skin epidermal cell line JB6 C141 dramatically decreases cell proliferation rate. It has also been reported that over-expression of paxillin in lung cancer H522 cells increase tumor microvessel density, whereas reduction of paxillin by thiolutin is related to the inhibition of HUVEC adhesion to vitronectin and impairment of angiogenesis. These reports support the notion that in our HNSCC tumor model, decreased expression of paxillin caused by MPS-1 over-expression may play a role in MPS-1-induced tumor inhibition.

### MPS-1 Protein is Highly Expressed in HNSCC.

Our research has demonstrated that MPS-1 can be detected in the sera of patients with HNSCC and appears to be a promising marker of presence of HNSCC. The levels of MPS-1 decrease with clinical tumor eradication (FIG. 2). With the thought that MPS-1 was a putative target to which to direct potential new therapy, we investigated a new class of molecules as potential novel chemotherapy for HNSCC. We created a model with over-expression of MPS-1. The thought was that with an over-expression of a putative target (MPS-1), we might see an enhanced response to drug treatment in subsequent experiments.

UMSCC-1 head and neck squamous carcinoma cells were transfected with either a control plasmid (pIRES2-EGFP, 5308 base pairs) or a plasmid containing cDNA for MPS-1 tagged with His(6) at the C-terminal. The pIRES2-EGFP vector was originally created by Clontech (Mountain view, Calif.), but has been discontinued for a while. A map can be found online: http://www.addgene.org/pgvec1?f=c&cmd=showvecinfo&vectorid=375). MPS-1 is located at EcoRI (629) restriction site with His at the C-terminal end.

After several cell sortings by flow cytometry, more than 95% of cells expressed green fluorescent protein (GFP). The cell lysates were analyzed by Western blot, confirming that MPS-1 protein was highly expressed in UMSCC-1/MPS-1 cells compared with UMSCC-1/control cells (FIG. 3a). To determine the sub cellular location of MPS-1, we tested for the presence of MPS-1 in the cytosol and nuclear extractions. We found that MPS-1 is not only a cytosolic protein, but also resides in the nucleus (FIG. 3b). Further, dot blotting analysis on the conditioned medium from the cultured cells showed that MPS-1 protein with His tag was secreted into the conditioned medium from UMSCC-1/MPS-1 cell culture (FIG. 3C), which is consistent with our previous findings that MPS-1 could be quantitatively detected in the serum from HNSCC patients.

### Over-Expressed MPS-1 Inhibits Cancer Development in UMSSC-1 Cells In Vitro.

As shown in FIG. 4a, the growth of UMSCC-1 cells expressing high level MPS-1 was significantly inhibited compared with control cells (P<0.01). Our data suggest that MPS-1 might not cause HNSCC and other malignancies. By contrast, high levels of MPS-1 induced during rapid HNSCC tumor cell proliferation inhibited tumor growth, which may be accomplished through MPS-1's extra-ribosomal functions, such as regulating gene expression, cell proliferation, and/or angiogenesis. This might be an attempt by the tumor cells or the host to down-regulate the tumor. In addition, MPS-1 expression in breast cancer cell line MDA-468 was induced by TGF-β1, which reduces MDA-468 cells' growth rate. This implies that MPS-1 may have an inhibitory role in regulating breast cancer cell growth as well.

### Over-Expressed MPS-1 Inhibits Cancer Development in UMSSC-1 Cells In Vivo.

To determine whether high level MPS-1 also inhibits tumor growth in vivo, animal experiments were conducted. In one experiment, 42 days after injection of UMSCC-1/MPS-1 cells into the left flank of nude mice, none of the mice formed tumors (0 out of 6). In contrast, all mice (6 out of 6) receiving UMSCC-1/control cell inoculation developed, tumors. In a repeat of the same experiment, all mice (n=6) injected with UMSCC-1/MPS-1 cells developed tumors; however, the onset of tumor development was delayed compared with UMSCC-1/control cells (28 days for UMSCC-1/MPS-1 cells vs. 14 days for UMSCC-1/control cells). Monitoring the tumor volume showed that UMSCC-1/control cells grew faster than UMSCC-1/MPS-1 cells in vivo (P<0.01) (FIG. 4B). Upon sacrifice (day 47), the mean weights of the primary tumors were 0.17 g (n=6) and 0.68 g (n=4) for UMSCC-1/MPS-1 and UMSCC-1/control tumors, respectively (p=0.016) (FIGS. 4C and D). Collectively, these data demonstrate that MPS-1 is a strong inhibitor of HNSCC cell growth both in vitro and in vivo.

### Mechanism of MPS-1 Over-Expression Altering Tumor Cell Growth in UMSCC-1 Cancer Cell Line.

Once we established that MPS-1 over-expression delays the onset of HSNCC tumor formation and slows tumor growth in mice, we wanted to determine how MPS-1 alters tumor growth. MPS-1 protein contains one zinc finger domain of the C₄ type and a cyclic AMP-responsive element; therefore, it may be involved in DNA repair and cell-cycle control. To investigate the effect of MPS-1 over-expression on cell-cycle control in HNSCC UMSCC-1 cells, we analyzed the DNA cell cycle by propidium iodide staining and flow cytometry on cultured cells. We showed that over-expression of MPS-1 caused cell-cycle arrest, indicated by an increase of cells in G0/G1 phase (UMSCC-1/control: 42.76% vs. UMSCC-1/MPS-1: 51.37%) and a decrease of cells in S phase (UMSCC-1/control: 28.42% vs. UMSCC-1/MPS-1: 19.12%). However, cell-cycle analysis did not detect a significant difference regarding the number of apoptotic cells in the sub-G0 phase (FIG. 5A).

We further immunostained tumor sections for Ki-67, which is a proliferation marker present during all active phases of the cell cycle, but absent in resting (G0) cells. Our data showed that 18.73±6.23% of UMSCC-1/control tumor-cell nuclei were positive for Ki-67, where only 5.13±2.94% of UMSCC-1/MPS-1 tumor-cell nuclei were Ki-67 positive (p=0.0003) (FIGS. 5B and C). In addition, we studied the apoptotic tumor cells by TUNEL staining on tumor sections and found very few TUNEL-positive cells for either UMSCC-1/control or UMSCC-1/MPS-1 tumors (FIG. 6). Together, this indicates that over-expression of MPS-1 protein in UMSCC-1 cells results in growth arrest rather than apoptosis, and suppresses tumor cell proliferation, which in part, contributes to the impaired tumor cell growth observed both in vivo and in vitro.

It has been shown that tumor angiogenesis is a major factor for growth of tumors, and this is a target of many new chemotherapeutic agents. Therefore, we decided to investigate whether the inhibition of the growth of the tumors formed by UMSCC-1/MPS-1 cells was related to altered tumor angiogenesis. We determined the mean micro-vessel density (MVD) by counting positively stained cells for CD34 antibody in five non-overlapping areas of each tumor section. Our data showed that tumors formed by UMSCC-1/MPS-1 cells had lower MVD (24.5±6.7/field) than those formed by UMSCC-1/control cells (69.2±16.4/field) (P=0.0003) (FIG. 7). These results suggest that the suppression of tumor angiogenesis is another contributory factor to the impaired growth of HNSCC tumor cells that over-express MPS-1 protein.

Paxillin Level is Reduced in UMSCC-1 Cells with Over-Expression of MPS-1.

Because MPS-1 contains a of zinc finger domain of C₄ type and is located in the nucleus, suggesting gene expression involvement, we performed gene microarray analysis and found that paxillin mRNA levels decreased by 24-45% in the UMSCC-1/MPS-1 cell line and 61-66% in UMSCC-1/MPS-1 tumors compared with controls. To determine if these results were reflected in protein levels, Western blotting was performed and confirmed that paxillin is significantly lower in both UMSCC-1/MPS-1 cell line and tumors than their controls (p=0.003) (FIGS. 8A, B and C). These findings indicate that MPS-1 down-regulates paxillin mRNA transcription and, thus, paxillin protein expression in tumor cells.

Although the mechanisms by which elevated MPS-1 decreases HNSCC tumor growth are still not entirely clear, we examined tumor cell apoptosis, cell proliferation, and tumor angiogenesis—aberrations of which have been reported to be closely related to tumor cell growth in vitro and in vivo. We find that over-expression of MPS-1 neither suppresses nor induces tumor cell apoptosis, rather it causes tumor cell G0/G1 arrest and reduces the cell proliferation rate. Because MPS-1 is a DNA-binding protein and may act as a component of a DNA repair system or induce other genes related to DNA repair, elevated MPS-1 protein may enhance recognition of damaged DNA in cancer cells, resulting in cell cycle arrest. This notion is supported by the finding that G1 arrest happens after DNA damage by gamma irradiation in hematopoietic cells lacking endogenous p53 but transfected with wild-type p53. P53 works to recognize damaged DNA and initiate repair processes as a cell-cycle checkpoint. Additionally, we found that tumor micro-vessel formation is greatly impaired in tumors formed by MPS-1 over-expressing UMSCC-1 cells. We do not currently know how MPS-1 impairs tumor angiogenesis; however, we believe secreted MPS-1 may play a role in suppressing tumor angiogenesis by interacting with extracellular matrix molecules associated with angiogenesis or directly functioning on the blood vessel-forming cells.

### Continuous MPS Over-Expression as a Means of Therapy.

MPS-1 expression vectors constantly over produce MPS-1 which may explain is tumor suppression phenotype. This could be mimicked pharmacologically as a potential application of this discovery for clinical oncology. An osmotic min-pump was placed in 2 groups of mice containing saline (control) or synthetic MPS-1. The MPS-1 treatment group demonstrated suppression of UMSSC-1 tumor growth in our nude mouse model (FIG. 9).

### Other Cancer Cell Lines.

In other experiments, we found the same inhibitory potential of MPS-1 on multiple myeloma (CAG) (FIG. 10) and lung cancer cells (H1299) (FIG. 11) growth in nude mice, suggesting the inhibitory function of MPS-1 is not limited to HNSCC.

### SUMMARY OF THE INVENTION

There is little evidence about the role of over-expressed r-proteins in regulating tumor cell behaviors. We provide strong evidence for the first time that over-expression of MPS-1 (a ribosomal protein S27, (RPS27)) can exert potent growth suppression in HNSCC tumor cells both in vivo and in vitro. In addition, MPS-1 can decrease mRNA and protein expression of paxillin, which is related to cell proliferation and angiogenesis regulation. This same phenotype of suppression has been demonstrated in 2 additional types of cancer implying that this effect might be present among many or all malignancies. This finding is a non-intuitive finding with respect to other patents on MPS and, therefore, should be considered novel and unique.

### BRIEF DESCRIPTION OF THE DRAWINGS

FIG. 1. MPS-1 levels measured in the serum of head and neck cancer and control patients.

FIG. 2. MPS-1 levels in the serum of HNSCC patients which were successfully treated or NOT of their cancer.

FIG. 3. Western blots demonstrating successful over-expression of MPS-1 by expression vector. A) Transfected cell lysates of control and MPS-1-cell line probed for MPS-1, B) location of MPS-1 in nucleus and cytosol of control and MPS-1 cells, C) MPS-1 presence in conditioned media of MPS-1 cells and control.

FIG. 4. MPS-1 suppresses HNSCC. High level of MPS-1 protein inhibits UMSCC-1 cells growth in vitro and in vivo. A) control and MPS-1 cell growth curves in culture, B) control and MPS-1 tumor growth curves in nude mice, C) representative nude mice from panel B, and D) tumor weights of control and MPS-1 tumors at end of experiment.

FIG. 5. Mechanism studies to explain MPS-1 and tumor suppression. A) Cell cycle plot of control and MPS-1 cells, B) histology of control and MPS-1 tumors to the proliferative marker Ki-67, c) quantification by percentage of control and MPS-1 Ki-67 staining.

FIG. 6. Apoptotic study of MPS-1 over-expression.

FIG. 7. Microvessel density and MPS-1 over-expression. A) Histology from control and MPS-1 tumors and B) quantification of microvessel in control and MPS-1 tumors.

FIG. 8. MPS-1 effect on paxillin expression. A) Suppression of paxillin expression in MPS-1 vs. control cells, B) suppression of paxillin in MPS-1 vs. control tumors, and C) paxillin standardized to actin is suppressed in MPS-1 tumors vs. control.

FIG. 9. Continuous delivery of MPS-1 or saline in nude mice containing HNSCC tumors. A) Saline, B) MPS-1.

FIG. 10. Over-expression of MPS in multiple myeloma cell line (CAG). A) Control, B) MPS-1 over-expressers.

FIGS. 11A and B. Over-expression of MPS in lung cancer cell line (H1299). A) Control, B) MPS-1 over-expressers.

### DESCRIPTION OF THE PREFERRED EMBODIMENT

Although any of the applications of MPS-1 therapy are likely to work, out preferred embodiment of this invention would be administration of MPS-1 peptide to the tumor through direct delivery (i.e., injection, electroporation) or systemically (i.e., basal drug therapy, nano-particle delivery).

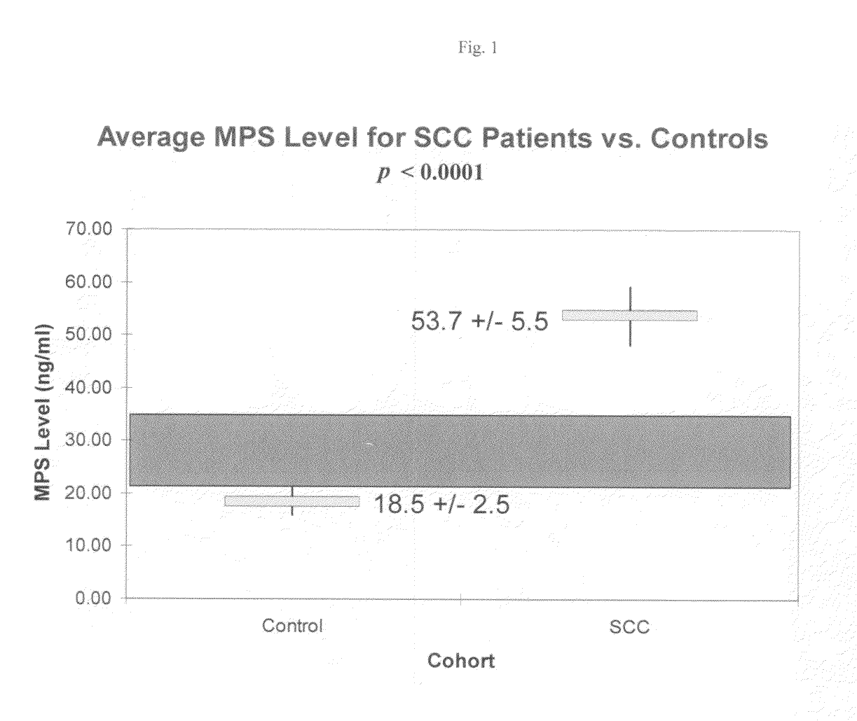

FIG. 1. MPS-1 levels measured in the serum of head and neck cancer and control patients. The amount of measured MPS by radioimmunoassay varies significantly between patients with and without head and neck cancer. Shaded area is range of normal MPS-1 levels. Stack, BC, World Journal of Surgical Oncology 2004.

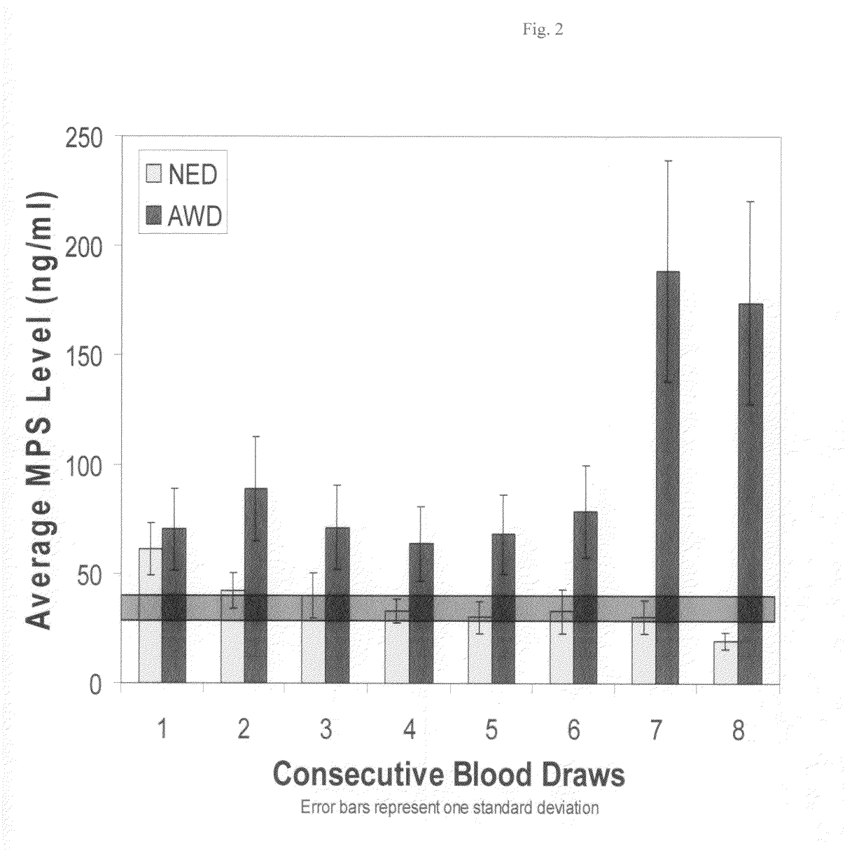

FIG. 2. MPS-1 levels in the serum of HNSCC patients which were successfully treated or NOT of their cancer. Differentiation of patients treated for HNSCC with and without clinically recurrent HNSCC using serum detection of MPS. NED=no evidence of disease. AWD=alive with disease. Shaded area is range of normal MPS-1 levels.

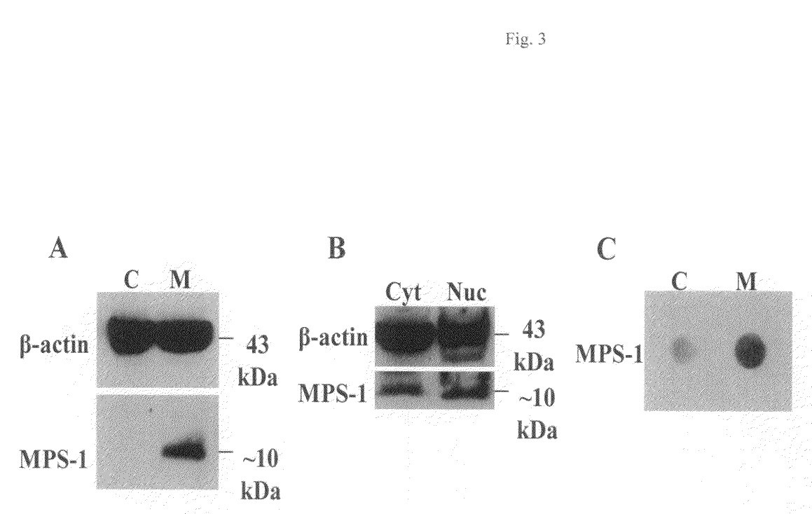

FIG. 3. Western blots demonstrating successful over-expression of MPS-1 by expression vector. Exogenous MPS-1 protein is present in both the UMSCC-1 cells and the conditioned media. A, MPS-1-transfected (M) cells express the protein in cell lysates. Western blots were probed with a monoclonal anti-His antibody that recognizes His tagged MPS-1 protein (˜10 kDa). MPS-1 protein with His tag was detected only in UMSCC-1/MPS-1 cells (M) rather than in UMSCC-1/control cells (C). B, In UMSCC-1/MPS, MPS-1 is located in not only the cytosol (Cyt) but also the nucleus (Nuc). C, Dot blotting by using the same anti-His antibody showed that His-tagged MPS-1 was detected in the conditioned media of UMSCC-1/MPS-1 cells (M), but not the control cells (C).

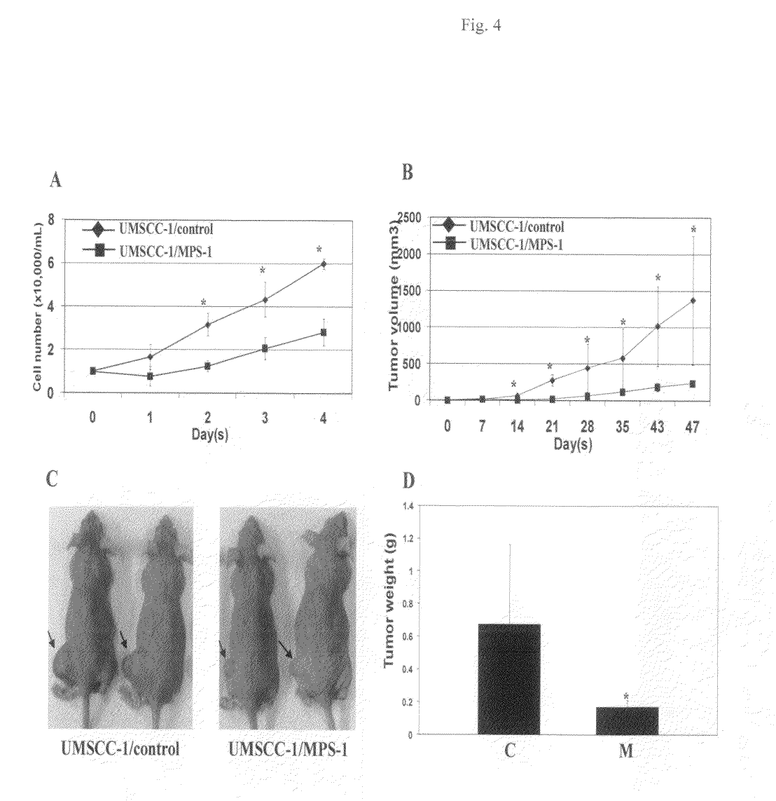

FIG. 4. MPS-1 suppresses HNSCC. High level of MPS-1 protein inhibits UMSCC-1 cells growth in vitro and in vivo. A, control-transfected (♦) or MPS-1-transfected (▪ cells (1×10⁴ cells/mL) were plated in 12-well plate and counted on four consecutive days (*, p<0.01). bars, SD. B, 2×10⁶ cells was injected subcutaneously in the left flank in male nude mice, tumor size was measured every week, and tumor volume was calculated. (*, p<0.01 for control versus MPS-1). bars, SD. C, representative tumors in the mice were shown upon sacrifice (arrow, tumor). D, upon sacrifice, tumors were harvested and weighed. Columns, mean tumor weight; bars, SD. (*, p=0.016. C: control; M: MPS-1).

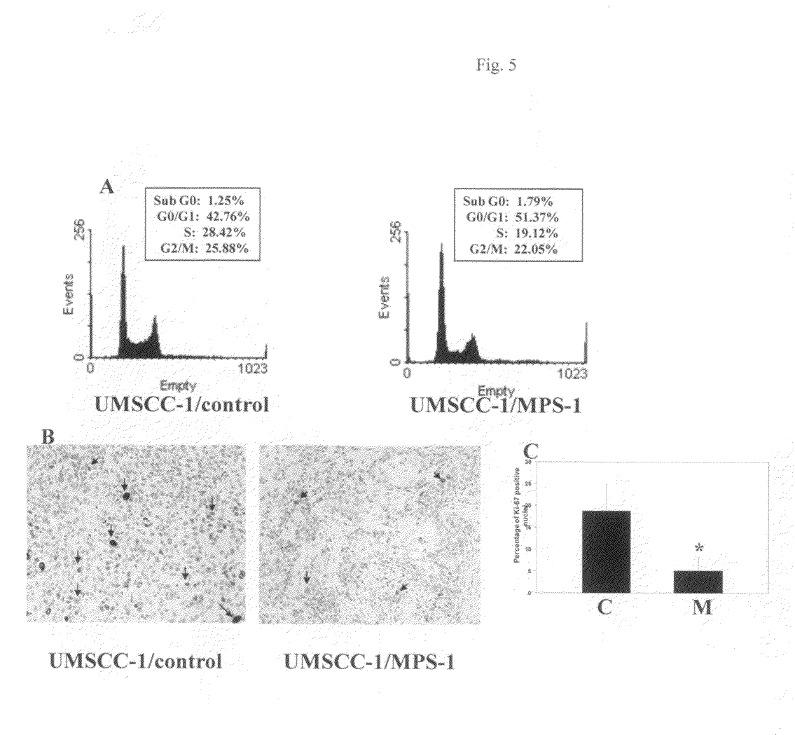

FIG. 5. Mechanism studies to explain MPS-1 and tumor suppression. A, DNA cell cycle assay was performed by PI staining and flow cytometry. B, nuclei were stained brown (arrows) with antibody to human Ki-67 (magnification, xl 00). C, percentage of nuclei positively stained with Ki-67 antibody. Columns, average percentage of Ki-67 positive nuclei in five microscopic fields (vertical axis); bars, SD. (*, p=0.0003. C: control; M: MPS-1).

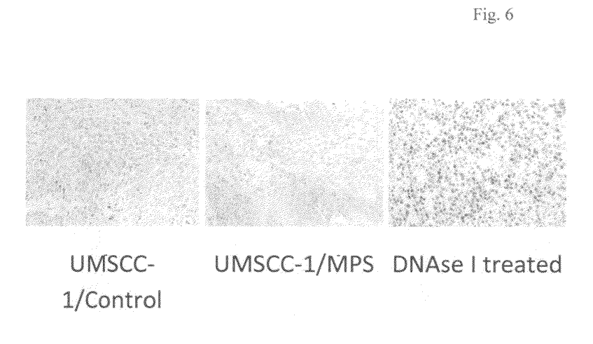

FIG. 6. Apoptotic study of MPS-1 over-expression. Apoptosis assay on tumor tissues by TUNEL staining (magnification, ×100). Few cells are positively stained in both negative control and MPS-1 tumors. The positive control slide was treated with DNase I before staining to create DNA strand breaks.

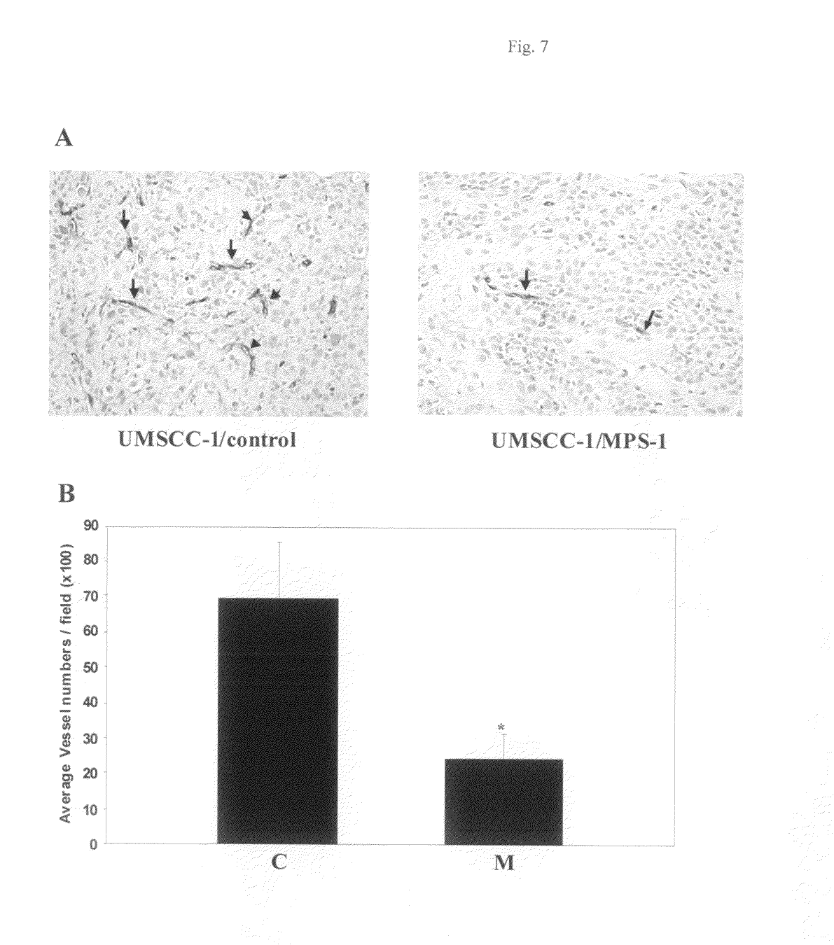

FIG. 7. Microvessel density and MPS-1 over-expression. Increased MPS-1 is associated with decreased micro-vessel density. A, tumor micro-vessel was stained with antibody to mouse CD34 (arrow, micro-vessel). B, number of vessels was counted. Columns, average vessel numbers in five microscopic fields (magnification, ×100); bars, SD. (*, p=0.0003 for control tumors (C) vs. MPS-1 tumors (M)).

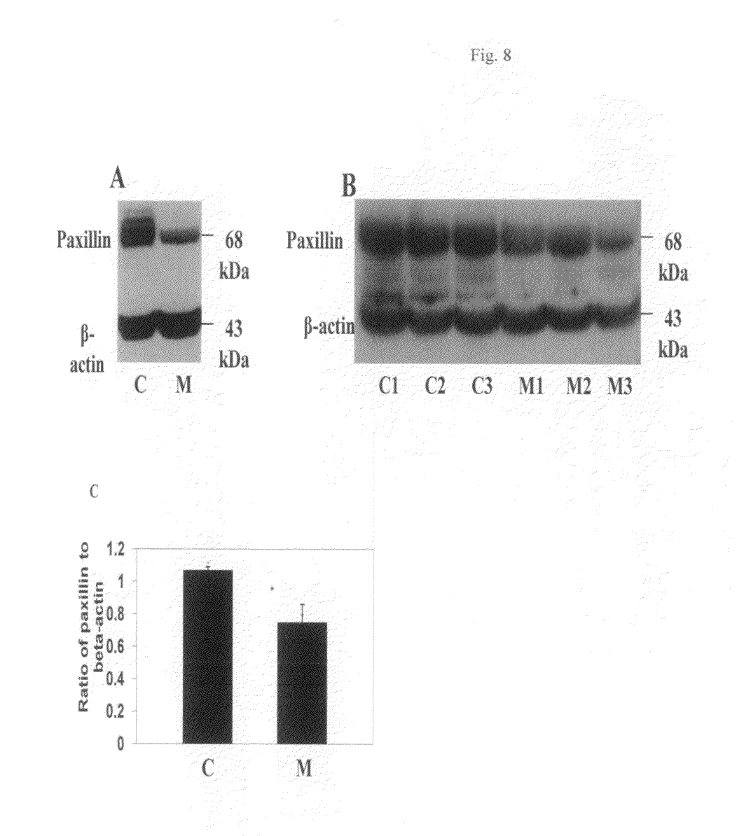

FIG. 8. MPS-1 effect on paxillin expression. A, Western blotting showed paxillin protein expression was suppressed in MPS-1 transfected UMSCC-1 cell line (M) compared to the control cell line (C). B, paxillin protein expression was lower in the tumors formed by UMSCC-1/MPS-1 cells (C1, C2, C3: tumors from three control mice; M1, M2, M3: tumors from three MPS-1 mice). C, the ratio of the intensity of the protein band of paxillin to β-actin by Western blotting was calculated by Image J software. Columns, average ratio; bars, SD. (*,p=0.003 for control tumors vs. MPS-1 tumors).

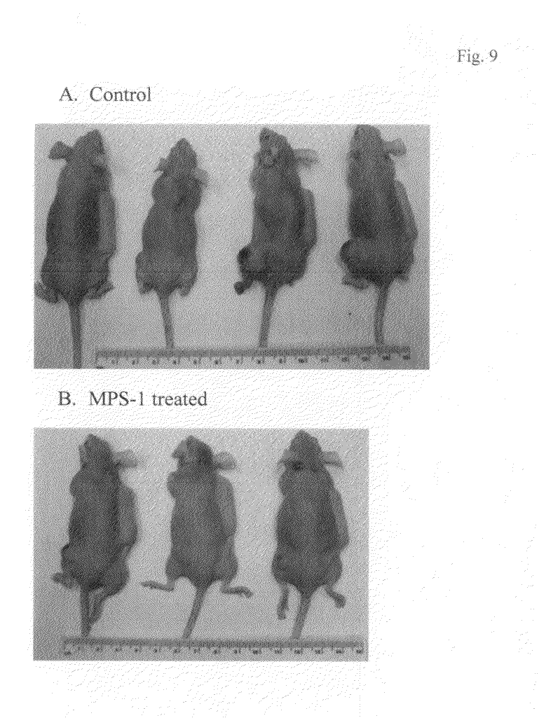

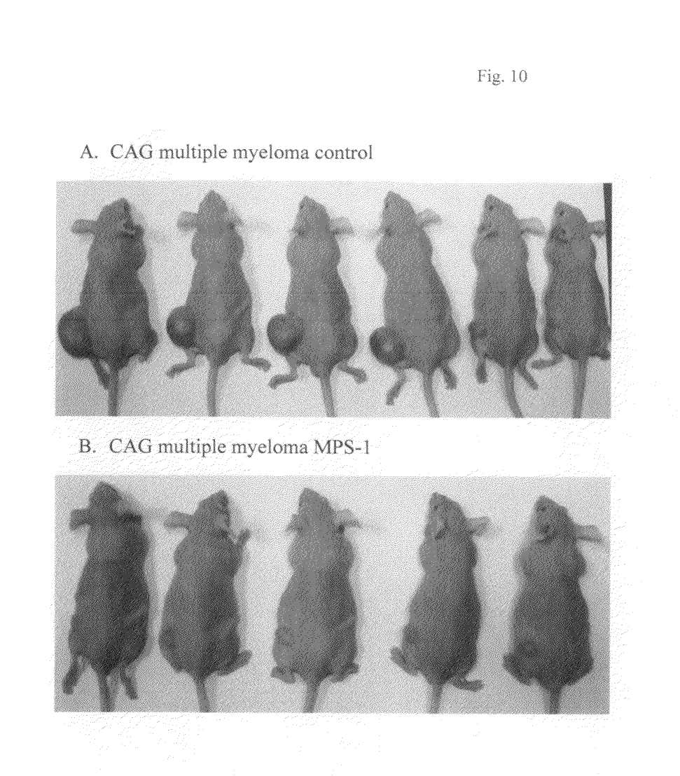

FIG. 9. Continuous delivery of synthetic MPS-1 in nude mice containing UMSCC-1 tumor xenograft control group (A) above and treated group (B) below. Flank cylindrical subcutaneous implant is the osmotic drug pump. FIG. 10. Over-expression of MPS in multiple myeloma cell line (CAG). Two million cells (either CAG-Control (A) or CAG-MPS-1-His (B)) were subcutaneously injected into nude mice. Five weeks after injection, mice were sacrificed and tumors were harvested. Tumors formed by CAG-MPS-1-His cells (B) are significantly smaller than the control ones (A). Picture after 5 weeks of tumor growth.

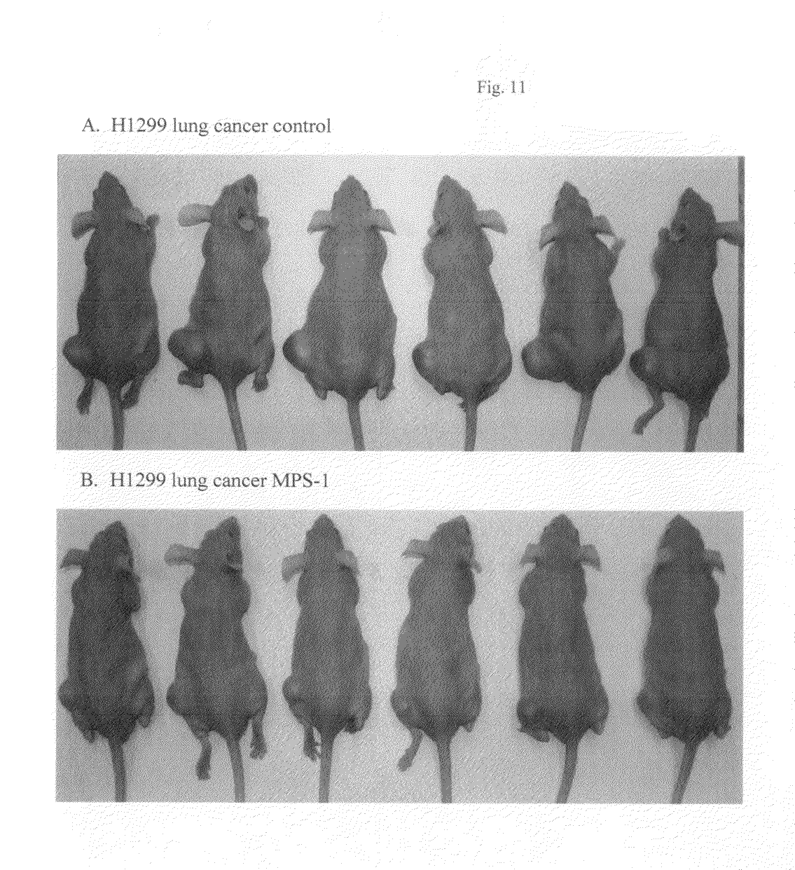

FIG. 11. Over-expression of MPS in lung cancer cell line (HI 299). Two million cells (either H1299-Control (A) or H1299-MPS-1-His (B)) were subcutaneously injected into nude mice. Five weeks after injection, mice were sacrificed and tumors were harvested. Tumors formed by H1299-MPS-1-His cells (B) are significantly smaller than the control ones (A). Picture after 5 weeks of tumor growth.

### EXAMPLES

### Dai Y, Pearson S, Cross D, Stack Jr B C, Int. J. Oncology 2009 In Press

Cell line and tissue culture. Human head and neck squamous cell carcinoma UMSCC-1 cells, kindly provided by Dr. T. E. Carey at the University of Michigan (Ann Arbor, Mich.), were cultured in Dulbecco's Modified Eagle's Medium (DMEM, Gibco, Grand Island, N.Y.), supplemented with 10% fetal bovine serum, 100 units/ml penicillin, and 100 μg/ml streptomycin sulfate and maintained at 37° C. in a cell culture incubator containing 5% CO₂.

Plasmid construction and transfection. The full-length fragment of human MPS-1 gene was amplified by RT-PCR from UMSCC-1 cells and the stop codon TAA was replaced with codon GCA encoding alanine. The fragment was inserted into plasmid pGS-21a (GenScript, Piscataway, N.J.), from which the new MPS-1 gene fragment with 6×His at C-terminal was obtained by PCR. Via the shuttle vector pGEM-T vector (Promega, Madison, Wis.), the new MPS-1-His fragment was sub cloned into the expression vector pIRES2-EGFP, kindly provide by Dr. Ralph D. Sanderson at University of Alabama at Birmingham (Birmingham, Ala.), with T4 ligase, and the insert was confirmed by sequencing.

10 μg of either vector pIRES2-EGFP/MPS-1 or the empty vector pIRES2-EGFP containing no insert as a control was transfected into the UMSCC-1 cells using Lipofectamine 2000 (Invitrogen, Carlsbad, Calif.) and Opti-MEM I (Invitrogen) per the manufacturer's instructions. The successfully transfected UMSCC-1 cells were sorted out by Flow Cytometry using GFP as marker. Before the further in vitro and in vivo experiments, the percentage of GFP positive cells was more than 95% as determined by Flow Cytometry.

Western blotting and dot blotting. Either UMSCC-1/control or UMSCC-1/MPS-1 cells in culture (2×10⁶) were pelleted by centrifugation, rinsed with ice-cold PBS, and lysed at room temperature in 200 μls of M-PER™ tissue protein extraction reagent (Pierce, Rockford, Ill.) added with Halt™ Protease Inhibitor Cocktail (Pierce). Lysates were centrifuged at 14,000×g for 15 min at room temperature. 20 μls of the lysates were loaded onto NuPAGE 4-12% Bis-Tri gels (Invitrogen) for electrophoresis, transferred to a nitrocellulose membrane, and probed with monoclonal mouse antibodies against either His(6) (GenScript) at 1:1000 dilution, paxillin (Clone 5H11) (Lab Vision, Fremont, Calif.) at 1:200 dilution or beta-actin (C4) (Santa Cruz Biotechnology, Santa Cruz, Calif.) at 1:1000 dilution. The blot was incubated with a horseradish peroxidase (HRP)-conjugated rabbit anti-mouse IgG (AnaSpec, San Jose, Calif.) and the protein was visualized by using PIERCE ECL Western Blotting Substrate kit (Pierce).

To examine paxillin expression in the tumor tissues, total proteins were extracted from the tumor tissues with T-PER™ tissue protein extraction reagent (Pierce) added with Halt™ Protease Inhibitor Cocktail (Pierce). The presence of paxillin was detected by Western Blotting as indicated above and the expression level was semi-quantitatively measured using NIH software Image J.

For dot blotting, the conditioned medium was collected from either UMSCC-1/control or UMSCC-1/MPS-1 cell culture for 48 hours with equal starting number of cells (5×10⁵/ml). 200 μls of conditioned medium was dot-blotted onto nitrocellulose membrane and the blot was probed with monoclonal mouse antibody against His(6) (GenScript) at 1:1000 dilution, followed by biotin-conjugated goat anti-mouse IgG secondary antibody (Vector Laboratories, Burlingame, Calif.). The protein dots were visualized by using PIERCE ECL Western Blotting Substrate kit (Pierce).

Cell fractionation. To examine the cellular localization of MPS-1 in the UMSCC-1/MPS-1 cells, sub cellular fractions were prepared using a Nuclear/Cytosol Fractionation Kit (BioVision, Mountain View, Calif.). The presence of MPS-1 was tested by Western Blotting as indicated above.

Tumor cell growth in vitro and in vivo. To assess the tumor cell proliferation in vitro, cells were plated in 12-well plates at a density of 1×10⁴ cells/mL with complete DMEM medium per well, in triplicate. The plates were incubated at 37° C. in 5% CO₂ for 0, 1, 2, 3, or 4 days. At each day, the cells were digested by trypsin-EDTA, cell number was counted and the cell density was calculated.

For the analysis of tumor growth in vivo, 2×10⁶ UMSCC-1/control cells or UMSCC-1/MPS-1 cells were harvested and re-suspended in 100 μls of ice-cold PBS and then subcutaneously injected into the left flank of 5-week old male athymic nude mice (Harlan Sprague Dawley, Indianapolis, Ind.). The tumor size was measured once a week and the tumor volume was calculated using the formula: length×(width)²×(Π/6) where the length was the longest dimension and width was the dimension perpendicular to length. Upon sacrifice, about week six after the injection, the tumors were harvested and weighed. Tumor tissue was then divided, and a portion was snap frozen in liquid nitrogen and then stored at −80° C. The remaining portion of tumor tissue was fixed in 10% formalin, pH 7.0, and embedded in paraffin for further analysis. Tissue sections were stained with hematoxylin and eosin for routine histological examination. All the procedures were approved by the Institutional Animal Care and Use Committee of the University of Arkansas for Medical Sciences and conformed to the guidelines established by the NIH.

Immunohistochemistry. To determine the microvessel density, formalin-fixed and paraffin-processed sections (5 μm) of tumors were de-paraffinized and rehydrated. Epitope retrieval was performed by steaming sections for 20 minutes in citrate buffer (pH 6.0). After quenching the endogenous peroxidase activity and blocking the non-specific binding sites, sections were incubated overnight with rat anti-mouse CD34 antibody (Serotec Ltd, Kidlington, Oxford, UK) at 1:120 dilution at 4° C., followed by incubation with biotin-conjugated rabbit anti-rat IgG secondary antibody (Vector Laboratories) at 1:100 dilution for 30 minutes at room temperature. After washing with PBS, the sections were incubated with ABC solution (Vector Laboratories) for 30 minutes at room temperature. The antibody staining was visualized by adding a 3,3′-diaminobenzidine (DAB) solution (Vector Laboratories) to the sections.

To evaluate tumor cell proliferation rate, the same immunohistochemistry procedure was performed except that the primary antibody was a monoclonal rabbit anti-human Ki-67 antibody (Clone SP6) (Lab vision) at 1:20 dilution and the secondary antibody was biotin-conjugated goat anti-rabbit IgG antibody (Vector Laboratories) at 1:100 dilution.

DNA cell cycle analysis. 1×10⁶ cells during log-phase growth were harvested and fixed overnight with ice cold 70% ethanol at 4° C. Cells were incubated with Propidium Iodide (PI) solution (0.1% BSA in PBS+0.1% Rnase+0.1 mg/ml PI) at room temperature in the dark for 30 minutes. After washing with PBS, DNA cell cycle was analyzed by FACSCalibur (BD, Franklin Lakes, N.J.).

Tumor TUNEL staining. To detect apoptosis on tumor sections, DeadEnd Colorimetric TUNEL System (Promega, Madison, Wis.) was applied. Per manufacture's manual, after being de-paraffinized, rehydrated and permeabilized by proteinase K, the sections were incubated with rTdTReactin Mix at 37° C. for 60 minutes, followed by incubation with Streptavidin HRP solution (1:500) at room temperature for 30 minutes. The staining was visualized by incubating the sections with DAB solution.

Gene array analysis. Total RNA was extracted from cell lines UMSCC-1/control, UMSCC-1/MPS-1, or the tumor tissues formed by these cells in the nude mice using Trizol Reagent (Invitrogen,), per manufacture's instruction. Gene expression profiling was performed with the Affymetrix U133 Plus 2.0 microarray platform (Affymetrix, Santa Clara, Calif.). Determination of mRNA levels was performed with Affymetrix microarray suite GCOS1.1 software.

Statistical analyses. All data are expressed as Mean±S.D. and the differences between the control group and MPS-1 group were calculated with the Student's t test. P<0.05 was set as statistical significance.

What we claim:

1. Over-expression of Metallopanstimulin (MPS-1) reduces tumor cell proliferation (defined as any malignancy or cancer) in vitro and its tumor size in vivo.

2. Administration of MPS-1 within a tumor will provide a tumor reducing therapy for cancer.

3. Systemic administration of MPS-1 will reduce tumor size.

4. Gene therapy which results in MPS-1 over-expression will reduce tumor size.

5. MPS-1 administration reduces paxillin expression/levels which, in turn, suppress cancer growth/burden.

6. MPS-1 administration reduces microvascular (blood vessel) density in cancer and is considered anti-angiogenic.

7. MPS-1 administration increases the shedding of CD138 from cancer cells.

8. MPS-1 administration decreased kappa chain immunoglobulin production/concentration from multiple myeloma.
